(19) United States
(12) Patent Application Publication
(10) Pub. No.: US 2017/0173330 A1
(43) Pub. Date: Jun. 22, 2017

(54) TRANSCRANIAL CURRENT STIMULATION (TCS) APPARATUS

(71) Applicant: Y-BRAIN INC., Daejeon (KR)

(72) Inventor: Kiwon LEE, Daejeon (KR)

(21) Appl. No.: 15/448,493

(22) Filed: Mar. 2, 2017

Related U.S. Application Data
(63) Continuation of application No. PCT/KR2015/007595, filed on Jul. 22, 2015.

(30) Foreign Application Priority Data
Sep. 3, 2014 (KR) 10-2014-0116681
Sep. 3, 2014 (KR) 10-2014-0116688

(51) Int. Cl. A61N 1/36; A61N 1/20; A61N 1/04

(52) U.S. Cl. CPC A61N 1/36025; A61N 1/0456; A61N 1/20

(57) ABSTRACT

Provided is a transcranial current stimulation apparatus. A transcranial current stimulation apparatus according to one embodiment of the present invention, which is put on or attached to the head of a user to apply an electrical stimulation to the brain, comprises: a first anode which comes into contact with a first part of the head and through which a first current flows; and a first cathode which comes into contact with a second part of the head and has a different amount of current from the first current.

### CROSS-REFERENCE TO RELATED APPLICATIONS

The present application is a continuation of International Patent Application No. PCT/KR2015/007595 filed on Jul. 22, 2015, which is based upon and claims the benefit of priority to Korea Patent Application No. 10-2014-0116681, filed on Sep. 3, 2014 and Korea Patent Application No. 10-2014-0116688, filed on Sep. 3, 2014. The disclosures of the above-listed applications are hereby incorporated by reference herein in their entirety.

### BACKGROUND

Embodiments of the inventive concept described herein relate to a transcranial current stimulation (tCS) apparatus, and more particularly, relate to a tCS apparatus in which an amount of current which flows through an anode differs from an amount of current which flows through a cathode.

It has been known that technology for brain electrical stimulation using tCS is effective to increase cognitive abilities and treat mental diseases such as depression and attention deficit hyperactivity disorder (ADHD).

After a first electrode which plays a role as an anode and a second electrode which plays a role as a cathode come into contact with a head of a user for tCS, a constant amount of current flows through the first and second electrodes to stimulate a brain of the user.

Herein, there is a difference between functions of the anode and the cathode used for tCS. For example, the anode may stimulate a brain function through stimulation, and the cathode may suppress the brain function.

Herein, according to conventional technology for brain electrical stimulation using tCS, there is a method of electrically stimulating the brain in a state where the same amount of current flows through the anode and the cathode. However, if the same amount of current flows through the anode and the cathode, brain stimulation may be achieved through only a network formed between the anode and the cathode at intervals of the brain of the user.

### SUMMARY

Embodiments of the inventive concepts provide a tCS apparatus for stimulating a brain of a user through various networks of an inner brain by having different amounts of current which flows through an anode and a cathode.

Embodiments of the inventive concept provide a tCS apparatus for being safely used in a user's daily life by a general user who does not have professional knowledge.

Embodiments of the inventive concept provide a tCS apparatus for having portability and wearability due to its simple configuration.

According to an aspect of an embodiment, a transcranial current stimulation (tCS) apparatus configured to be worn or attached to a head of a user to provide electrical stimulation a brain of the user may include a first anode configured to come into contact with a first part of the head such that a first current flows through the first anode and a first cathode configured to come into contact with a second part of the head such that a second current having a different amount of current from the first current flows through the first cathode.

According to another aspect of an embodiment, a transcranial current stimulation (tCS) apparatus configured to be worn or attached to a head of a user to provide electrical stimulation a brain of the user may include a first anode configured to come into contact with a first part of the head such that a first current flows through the first anode and a second anode configured to come into contact with a second part of the head such that a second current having a different amount of current from the first current flows through the second anode.

According to another aspect of an embodiment, a transcranial current stimulation (tCS) apparatus configured to be worn or attached to a head of a user to provide electrical stimulation a brain of the user may include a first cathode configured to come into contact with a first part of the head such that a first current flows through the first cathode and a second cathode configured to come into contact with a second part of the head such that a second current having a different amount of current from the first current flows through the second cathode.

According to another aspect of an embodiment, a transcranial current stimulation (tCS) apparatus configured to be worn or attached to a head of a user to provide electrical stimulation a brain of the user may include a first electrode configured to come into contact with a first part of the head such that a first current flows through the first electrode and a second electrode configured to come into contact with a second part of the head such that a second current having a different amount of current from the first current flows through the second electrode. The first current and the second current may differ from each other in phase or frequency.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram schematically illustrating a configuration of a transcranial current stimulation (tCS) apparatus according to an embodiment of the inventive concept;

FIG. 2 is a drawing illustrating a tCS apparatus of FIG. 1, worn or attached to a user;

FIG. 3 is a drawing schematically illustrating a tCS apparatus according to another embodiment of the inventive concept;

FIGS. 4 and 5 are drawings illustrating a tCS apparatus of FIG. 3, worn or attached to a user;

FIG. 6 is a drawing schematically illustrating a tCS apparatus according to another embodiment of the inventive concept;

FIG. 7 is a drawing illustrating a tCS apparatus of FIG. 6, worn or attached to a user;

FIG. 8 is a block diagram schematically illustrating a tCS apparatus according to another embodiment of the inventive concept;

FIG. 9 is a drawing illustrating a tCS apparatus of FIG. 8, worn or attached to a user;

FIG. 10 is a block diagram schematically illustrating a configuration of a tCS apparatus according to another embodiment of the inventive concept;

FIG. 11 is a drawing illustrating a tCS apparatus of FIG. 10, worn or attached to a user;

FIG. 12 is a drawing schematically illustrating a tCS apparatus according to another embodiment of the inventive concept;

FIG. 13 is a drawing schematically illustrating a tCS apparatus according to another embodiment of the inventive concept;

FIG. 14 is a drawing schematically illustrating a tCS apparatus according to another embodiment of the inventive concept;

FIG. 15 is a drawing schematically illustrating a tCS apparatus according to another embodiment of the inventive concept; and

FIGS. 16 and 17 are drawings illustrating a current of a tCS apparatus according to another embodiment of the inventive concept.

### DETAILED DESCRIPTION

Hereinafter, a description will be given in detail of exemplary embodiments of the inventive concept with reference to the accompanying drawings. Advantages, features, and methods of accomplishing the same will become apparent with reference to embodiments described in detail below together with the accompanying drawings. However, the inventive concept is not limited by embodiments disclosed hereinafter, and may be implemented in various forms. Rather, these embodiments are provided to so that this disclosure will be through and complete and will fully convey the concept of the invention to those skilled in the art, and the inventive concept will only be defined by the appended claims. Like reference denotations refer to like elements throughout the specification.

Unless otherwise defined herein, all terms (including technical and scientific terms) used in the specification may have the same meaning that is generally understood by a person skilled in the art. Also, terms which are defined in a dictionary and commonly used should be interpreted as not in an idealized or overly formal detect unless expressly so defined.

Terms used in the specification are used to describe embodiments of the inventive concept and are not intended to limit the scope of the inventive concept. In the specification, the terms of a singular form may include plural forms unless otherwise specified. The expressions “comprise” and/or “comprising” used herein indicate existence of one or more other elements other than stated elements but do not exclude presence of additional elements.

Transcranial current stimulation (tCS) in the specification may be any one of transcranial alternating current stimulation (tACS) or transcranial direct current stimulation (tDCS). Thus, if a tCS apparatus uses AC in embodiments of the inventive concept, it may be a tACS apparatus. If the tCS apparatus uses DC in embodiments of the inventive concept, it may be a tDCS apparatus.

Hereinafter, a description will be given of a tCS apparatus according to embodiments of the inventive concept with reference to drawings.

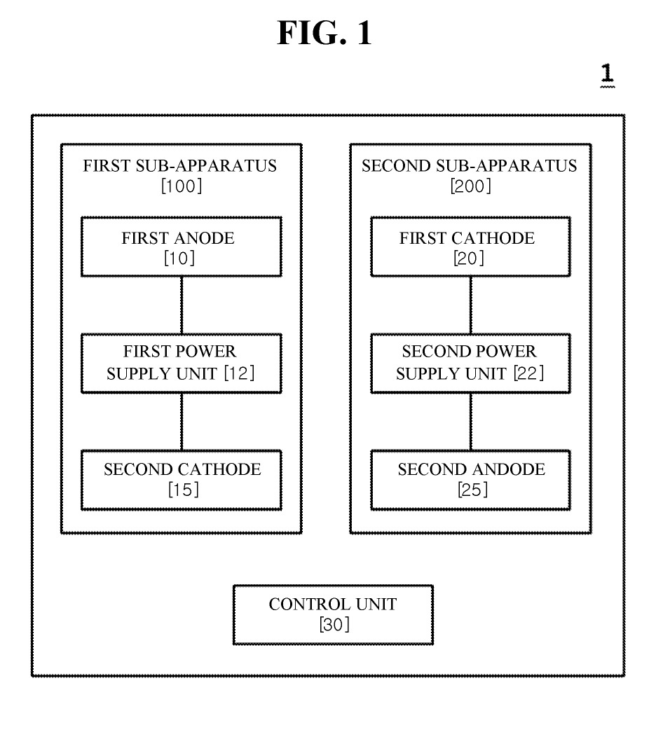

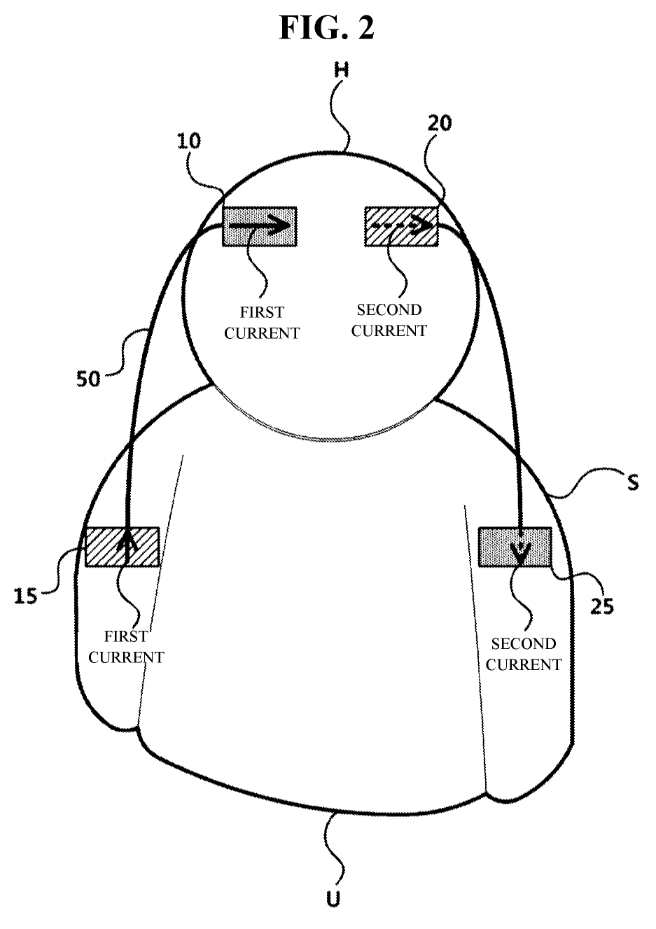

Referring to FIGS. 1 and 2, a description will be given of a tCS apparatus 1 according to an embodiment of the inventive concept. Referring to FIG. 1, a configuration of a tCS apparatus 1 according to an embodiment of the inventive concept is schematically illustrated. Referring to FIG. 2, a tCS apparatus of FIG. 1, worn or attached to a user, is illustrated. Herein, for convenience of description, the tCS apparatus 1 excluding a power supply unit and a control unit is shown in FIG. 2.

Meanwhile, the tCS apparatus 1 may be a tACS apparatus which uses AC or a tDCS apparatus which uses DC.

The tCS apparatus 1 according to an embodiment of the inventive concept may be worn or attached to a head H of a user U to provide electrical stimulation to a brain of the user U. For example, if the user U simply wears the tCS apparatus 1 on his or her head H, an anode and a cathode included in the tCS apparatus 1 may come into contact with a specific location of the head H and current flows in the head H of the user U through the anode and the cathode. Thus, electrical stimulation may be provided to a brain region to be targeted. Also, if the user U attaches the tCS apparatus 1 to a region where he or she wants to provide electrical stimulation, electrical stimulation may be applied to a brain region to be targeted.

In detail, referring to FIGS. 1 and 2, the tCS apparatus 1 may include a first anode 10 and a first cathode 20. The tCS apparatus 1 may further include first and second power supply units 12 and 22, a second cathode 15, a second anode 25, and a control unit 30. Herein, since the components shown in FIGS. 1 and 2 are not essential, the tCS apparatus 1 may be implemented to have more components than those shown in FIGS. 1 and 2 or have less components than those shown in FIGS. 1 and 2.

In detail, the tCS apparatus 1 may include first and second sub-apparatuses 100 and 200. The first sub-apparatus 100 and the second sub-apparatus 200 may be independent of each other. Herein, it may be understood that the meaning “be independent of each other” is the meaning that, since the first sub-apparatus 100 and the second sub-apparatus 200 do not share a power supply unit, each of them may adjust an amount of current which flows through each of them.

The first sub-apparatus 100 may include the first anode 10, the first power supply unit 12, and the second cathode 15. The second sub-apparatus 200 may include the first cathode 20, the second power supply unit 22, and the second anode 25. Herein, electrodes substantially used to provide electrical stimulation to a brain of the user U may be the first anode 10 and the first cathode 20. If the user U uses the tCS apparatus 1, the first anode 10 and the first cathode 20 may come into contact with the head H of the user U. The second anode 25 and the second cathode 15 may fail to be electrodes substantially used to provide electrical stimulation to the brain and may be located to come into contact with a body of the user U rather than the head H.

Since the first anode 10 and the first cathode 20 are connected to different power supply units and are included in different sub-apparatuses, an amount of current which flows through the first anode 10 are different from an amount of current which flows through the first cathode 20. Since current in the specification refers to DC or AC, the amount of current in the specification may mean, but is not limited to, amplitude of current. The amount of current in the specification may mean an effective value of current.

Herein, a description will be given in detail of each of the components included in the tCS apparatus 1 according to an embodiment of the inventive concept.

If the user U wears or attaches the tCS apparatus 1 on or to the head H, the first anode 10 may come into contact with a first part of the head H. A first current may flow through the first anode 10. The first anode 10 may stimulate the brain at a level corresponding to an amount of the first current to activate a brain function.

Meanwhile, the first current which flows through the first anode 10 may be DC or AC. Since the first current is weak current having, for example, an amount of current which is greater than 0 mA and is less than or equal to 2 mA, the first current may reduce the risk of side effects which may be generated if the tCS apparatus 1 according to an embodiment of the inventive concept is used.

If the user U wears or attaches the tCS apparatus 1 on or to the head H, the first cathode 20 may come into contact with a second part of the head H. The second part of the head H, which comes into contact with the first cathode 20, may be a part spaced apart from the first part of the head H, which comes into contact with the first anode 10. A second current may flow through the first cathode 20 and may have a different amount of current from the first current. For example, a level of the second current may be higher or lower than that of the first current. The first cathode 20 may suppress a brain function at a level corresponding to the amount of the second current.

Meanwhile, the second current which flows through the first cathode 20 may be DC or AC. Since the second current is weak current having, for example, an amount of current which is greater than 0 mA and is less than or equal to 2 mA, the second current may reduce the risk of side effects which may be generated if the tCS apparatus 1 according to an embodiment of the inventive concept is used.

The reason an amount of current which flows through the first anode 10 is different from an amount of current which flows through the first cathode 20 in the tCS apparatus 1 according to an embodiment of the inventive concept will be described hereafter. If the amount of current which flows through the first anode 10 is different from the amount of current which flows through the first cathode 20, strength of stimulation (activation) provided to the brain by the first anode 10 and strength of suppression provided to the brain by the first cathode 20 may be changed. The brain may be stimulated through various networks of an inner brain due to a difference between the strength of the stimulation (activation) and the strength of the suppression.

For example, if current which flows through an anode is the same as current which flows through a cathode in related art, brain stimulation may be achieved through a network having directionality of connecting the anode with the cathode. However, according to the tCS apparatus 1 according to an embodiment of the inventive concept, the amount of current which flows through the first anode 10 may be different from the amount of current which flows through the first cathode 20. Therefore, since brain simulation is achieved through another network of an inner brain rather than a network having directionality of connecting the first anode 10 with the first cathode 20, stimulation for the brain may be effectively achieved.

The first power supply unit 12 may be connected with the first anode 10 and the second cathode 15 to configure the first sub-apparatus 100 and may supply power such that the first current which flows through the first anode 10 differs from the second current which flows through the first cathode 20. For example, the first power supply unit 12 may be an AC power supply which may generate AC or a DC power supply which may generate DC.

The second cathode 15 may be electrically connected with the first anode 10 via the first power supply unit 12 such that the first current flows through the second cathode 15. Herein, since the second cathode 15 is not an electrode substantially used to provide electrical stimulation to the brain, it may be located to come into contact with the body of the user U except for the head H. For example, if the user U uses the tCS apparatus 1, the second cathode 15 may be attached to a shoulder S, an arm, or the like of the user U to be spaced apart from the brain. However, a location to which the second cathode 15 is attached is not limited thereto.

In other words, since the second cathode 15 is spaced apart from the head H and is attached to the user U, the second cathode 15 may fail to have an influence on brain stimulation.

The second power supply unit 22 may be connected with the first cathode 20 and the second anode 25 to configure the second sub-apparatus 20 and may supply power such that the second current which flows through the first cathode 20 differs from the first current which flows through the first anode 10. Particularly, the second power supply unit 22 may be a power supply which is independent of the first power supply unit 12. The second power supply unit 12 may be an AC power supply which may generate AC or a DC power supply which may generate DC.

The second anode 25 may be electrically connected with the first cathode 20 via the second power supply unit 22 such that the second current flows through the second anode 25. Herein, since the second anode 25 is not an electrode substantially used to provide electrical stimulation to the brain, it may be located to come into contact with a body of the user U rather than the head H. For example, if the user U uses the tCS 1, the second cathode 15 may be attached to a shoulder S, an arm, or the like of the user U and may be spaced part from the brain. However, a location to which the second cathode 15 is attached is not limited thereto.

In other words, since the second anode 25 is spaced apart from the head H and is attached to the user U, the second cathode 15 may fail to have an influence on brain stimulation.

The control unit 30 may control an overall operation of the tCS apparatus 1 and, for example, may control an amount of current which flows through each of the first anode 10 and the first cathode 20. If necessary, the control unit 30 may adjust a difference between an amount of the first current which flows through the first anode 10 and an amount of the second current which flows through the first cathode 20. For this purpose, each of the first and second sub-apparatuses 100 and 200 may include another additional component for current control.

Also, the control unit 30 may include a component for verifying whether the user U accurately wears or attaches the tCS apparatus 1. After verifying that the user U accurately wears or attaches the tCS apparatus 1, the control unit 30 may control the first and second power supply units 12 and 22 to provide electrical stimulation to the brain. For example, the control unit 30 may determine whether the user U accurately wears/attaches the tCS apparatus 1 by measuring impedance of a part where the tCS apparatus 1 comes into contact with the user U. The method of determining whether the user U accurately wears/attaches the tCS apparatus 1 is not limited thereto.

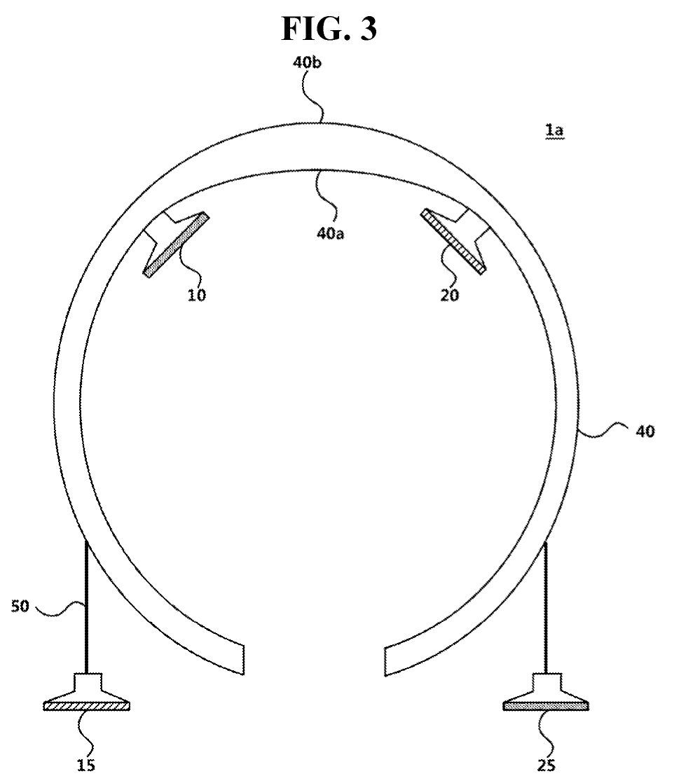

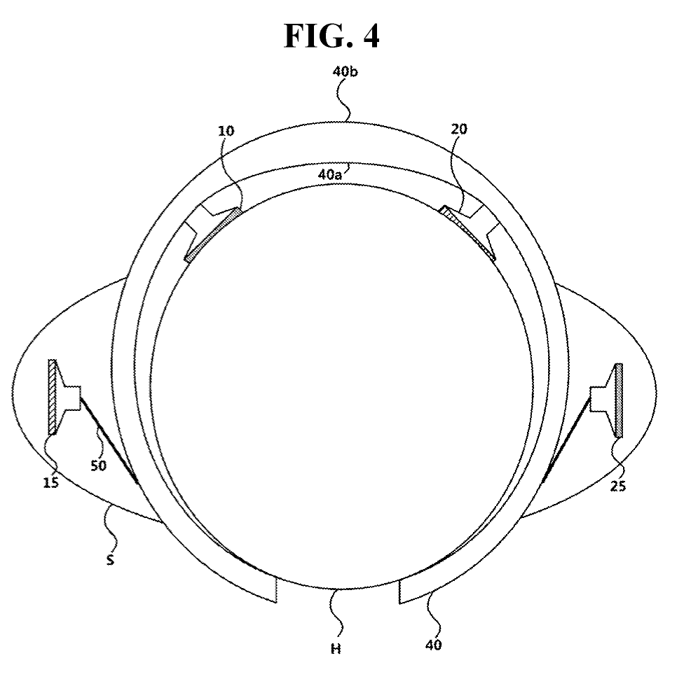

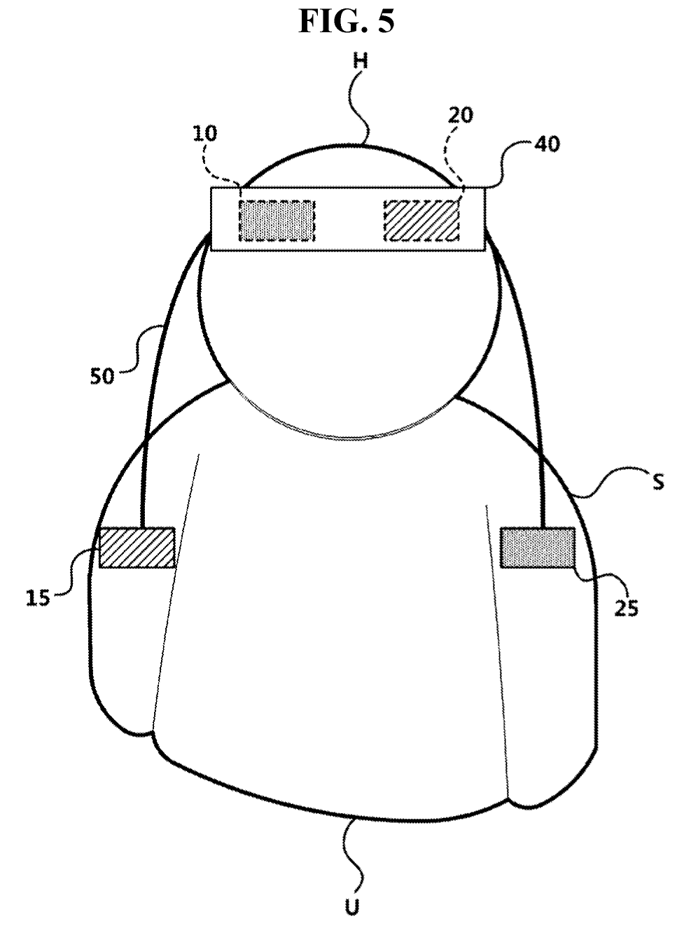

Hereinafter, a description will be given of a tCS apparatus 1a according to another embodiment of the inventive concept with reference to FIGS. 3 to 5. Herein, a difference between a tCS apparatus 1 according to an embodiment of the inventive concept and the tCS apparatus 1a according to another embodiment of the inventive concept is mainly described. It is apparent to those skilled in the art that technical features applied to the tCS apparatus 1 according to an embodiment of the inventive concept may be applied to the tCS apparatus 1a according to another embodiment of the inventive concept except for a described difference. Referring to FIG. 3, a drawing schematically illustrating a configuration of the tCS apparatus 1a according to another embodiment is shown. Referring to FIGS. 4 and 5, a drawing illustrating the tCS apparatus 1a of FIG. 3, worn on a user U, is shown.

Meanwhile, in another embodiment, the tCS apparatus 1a may be a tACS apparatus which uses AC or a tDCS apparatus which uses DC.

Referring to FIG. 3, the tCS apparatus 1a may include a frame 40. The frame 40 may be a framework of the tCS apparatus 1a. A first anode 10, a first cathode 20, a first power supply unit 12, a second power supply unit 22, the second anode 25, a second cathode 15, and a control unit 30 may be combined with the frame 40 or may be embedded in the frame 40. The frame 40 may include a first surface 40a faced with a head H of a user U and a second surface 40b opposite to the first surface 40a.

For example, the frame 40 may have a ring structure, one side of which is opened. If the frame 40 is worn on the head H of the user U, it may press the head H, thus being stably fixed to the head H without going down the head H. Thus, the user U wears the tCS apparatus 1a on the head H to use the tCS apparatus 1a. Herein, if the frame 40 has a shape which may be worn on the head H of the user U, the shape of the frame 40 is not limited thereto.

The first anode 10 and the first cathode 20 may be connected and fixed to the first surface 40a of the frame 40. Thus, if the user U wears the tCS apparatus 1a on the head H, the first anode 10 and the first cathode 20 may come into contact with the head H of the user U.

The second anode 25 and the second cathode 15 may be connected to the frame 40 via extension lines 50. Since each of the extension lines 50 is extended in length if necessary, the user U may easily attach the second anode 25 and the second cathode 15 to his or her shoulder S or arm.

Referring to FIG. 4, a plane view illustrating the tCS apparatus 1a worn on the user U is shown. Referring to FIG. 5, a perspective view illustrating the tCS apparatus 1a worn on the user U is shown. According to FIGS. 4 and 5, another embodiment of the inventive concept is exemplified as the user U wears the tCS apparatus 1a on the head H and attaches the second anode 25 and the second cathode 15 to the periphery of the shoulder S.

According to another embodiment of the inventive concept, the general user U who does not have professional knowledge may safely use the tCS apparatus 1a in his or her daily life. The tCS apparatus 1a may have excellent portability and wearability due to its simple configuration.

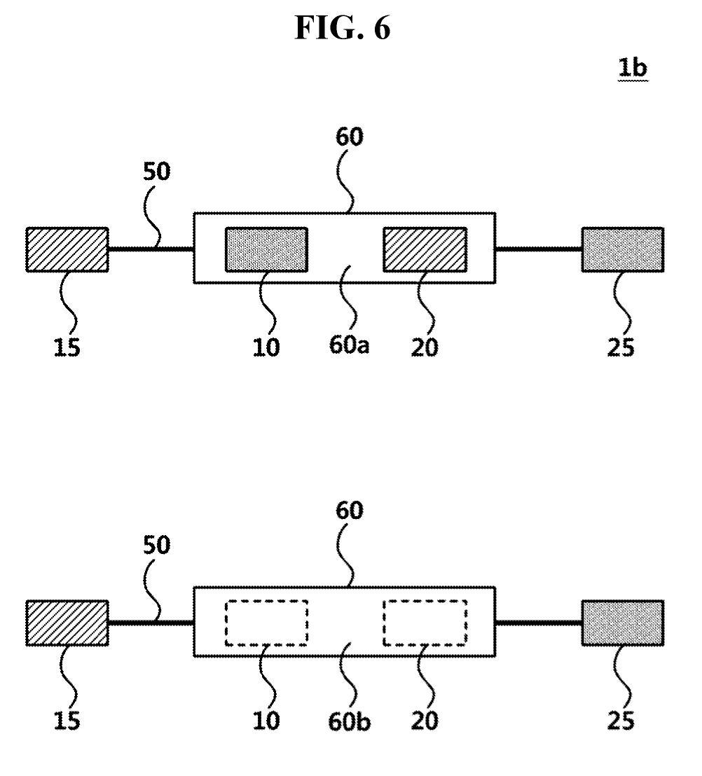

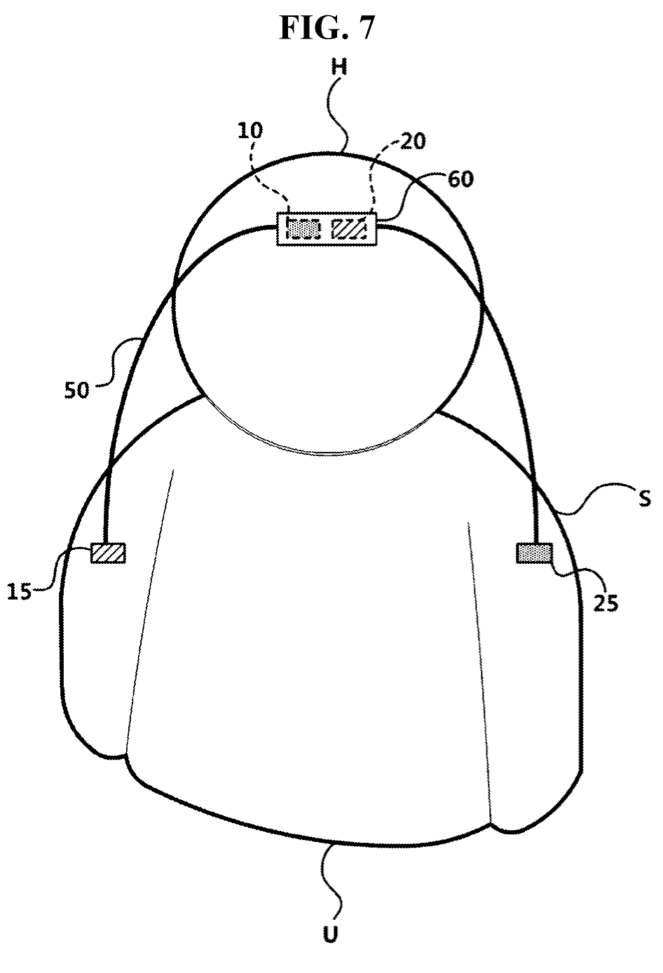

Hereinafter, a description will be given of a tCS apparatus 1b according to another embodiment of the inventive concept with reference to FIGS. 6 and 7. Herein, a difference between a tCS apparatus 1 according to an embodiment of the inventive concept and the tCS apparatus 1b according to another embodiment of the inventive concept is mainly described. It is apparent to those skilled in the art that technical features applied to the tCS apparatus 1 according to an embodiment of the inventive concept may be applied to the tCS apparatus 1b according to another embodiment of the inventive concept except for a described difference. Referring to FIG. 6, a drawing schematically illustrating a configuration of the tCS apparatus 1b according to another embodiment is shown. Referring to FIG. 7, a drawing illustrating the tCS apparatus 1b of FIG. 6, attached to a user U, is shown.

Meanwhile, in another embodiment, the tCS apparatus 1b may be a tACS apparatus which uses AC or a tDCS apparatus which uses DC.

Referring to FIG. 6, the tCS apparatus 1b may include a patch frame 60. The patch frame 60 may be a framework of the tCS apparatus 1b. A first anode 10, a first cathode 20, a first power supply unit 12, a second power supply unit 22, the second anode 25, a second cathode 15, and a control unit 30 may be combined with the patch frame 60 or may be embedded in the patch frame 60. Since the patch frame 60 may include a flexible board, if the tCS apparatus 1b is attached to a head H of a user U, it may be bent along a curve of the head H.

Meanwhile, the patch frame 60 may include a third surface 60a faced with the head H and a fourth surface 60b opposite to the third surface 60a. The third surface 60a of the patch frame 60 may be a surface where the first anode 10 and the first cathode 20 are located and may be a surface attached to the head H of the user U. There may be adhesive on the third surface 60a such that the patch frame 60 is attached to the head H. Thus, the user U may attach the tCS apparatus 1b to a part he or she wants, thus providing electrical stimulation to his or her brain

The second anode 25 and the second cathode 15 may be connected to the frame 40 via extension lines 50. Since each of the extension lines 50 is extended in length if necessary, the user U may easily attach the second anode 25 and the second cathode 15 to his or her shoulder S or arm.

Referring to FIG. 7, a perspective view illustrating the tCS apparatus 1b worn on the user U is shown. According to FIG. 7, another embodiment of the inventive concept is exemplified as the user U wears the tCS apparatus 1b on the head H and attaches the second anode 25 and the second cathode 15 to the periphery of the shoulder S.

According to another embodiment of the inventive concept, the general user U who does not have professional knowledge may safely use the tCS apparatus 1b in his or her daily life. The tCS apparatus 1b may have excellent portability and wearability due to its simple configuration.

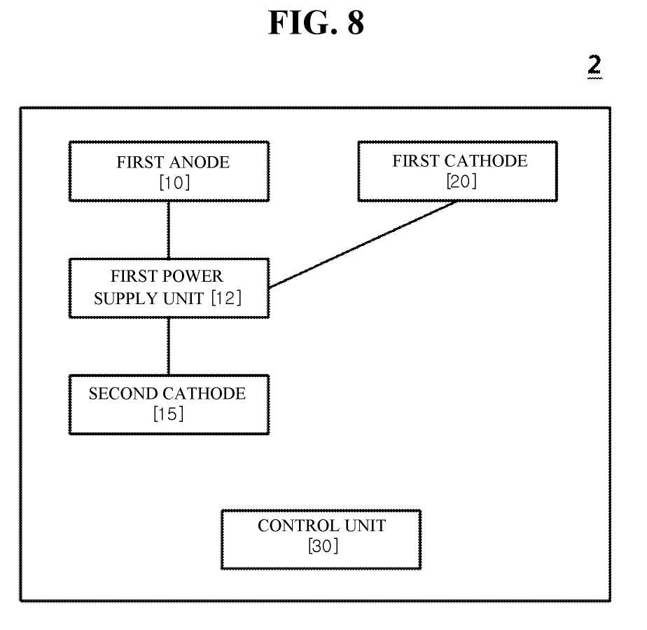

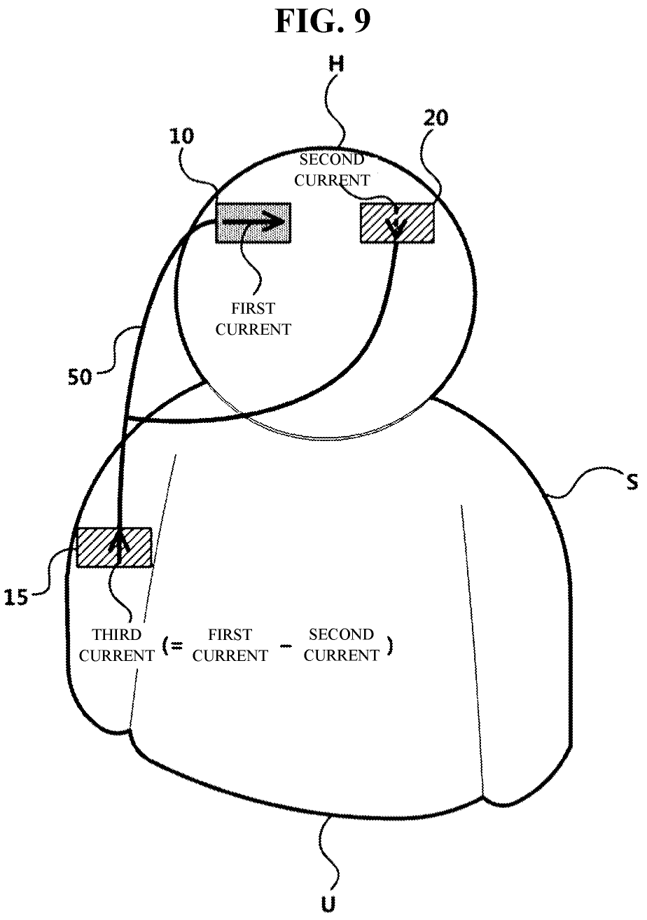

Hereinafter, a description will be given of a tCS apparatus 2 according to another embodiment of the inventive concept with reference to FIGS. 8 and 9. Herein, a difference between a tCS apparatus 1 according to an embodiment of the inventive concept and the tCS apparatus 2 according to another embodiment of the inventive concept is mainly described. It is apparent to those skilled in the art that technical features applied to the tCS apparatus 1 according to an embodiment of the inventive concept may be applied to the tCS apparatus 2 according to another embodiment of the inventive concept except for a described difference. Referring to FIG. 8, a drawing schematically illustrating a configuration of the tCS apparatus 2 according to another embodiment is shown. Referring to FIG. 9, a drawing illustrating the tCS apparatus 2 of FIG. 8, worn or attached to a user U is shown.

Meanwhile, in another embodiment, the tCS apparatus 2 may be a tACS apparatus which uses AC or a tDCS apparatus which uses DC.

Referring to FIG. 8, the tCS apparatus 2 may include a first anode 10, a first cathode 20, a second cathode 15, a first power supply unit 12, and a control unit 30. In other words, in the tCS apparatus 2 according to another embodiment of the inventive concept, the number of anodes may be different from the number of cathodes. For example, the tCS apparatus 2 may include the one anode 10 and the two cathodes 15 and 20. All of the first anode 10, the first cathode 20, and the second cathode 15 may be connected to the first power supply unit 12.

Meanwhile, in another embodiment, the tCS apparatus 2 may be a tACS apparatus which uses AC or a tDCS apparatus which uses DC.

Referring to FIG. 9, if the user U wears or attaches the tCS apparatus 2 on or to the head H, the first anode 10 may come into contact with a first part of the head H. A first current may flow through the first anode 10. If the user U wears or attaches the tCS apparatus 2 on or to the head H, the first cathode 20 may come into contact with a second part of the head H. A second current which has a different amount of current from the first current may flow through the first cathode 20.

Further, the second cathode 15 may be electrically connected with the first anode 10 and the first cathode 20 and, for example, may be located to come into contact with a body of the user U rather than the head H. A third current may flow through the second cathode 15. The third current may have the same amount of current as an amount of current in which an amount of the second current is excluded from an amount of the first current.

In some embodiments, the first cathode 20 and the second cathode 15 may include a current adjusting circuit (not shown) or a current limiting circuit (not shown) for adjusting/limiting an amount of current. Thus, an amount of current which flows through any one of the first cathode 20 and the second cathode 15 may be adjusted/limited by the current adjusting circuit/the current limiting circuit. The remaining amount of current rather than the adjusted/limited amount of current may flow through the other of the first cathode 20 and the second cathode 15.

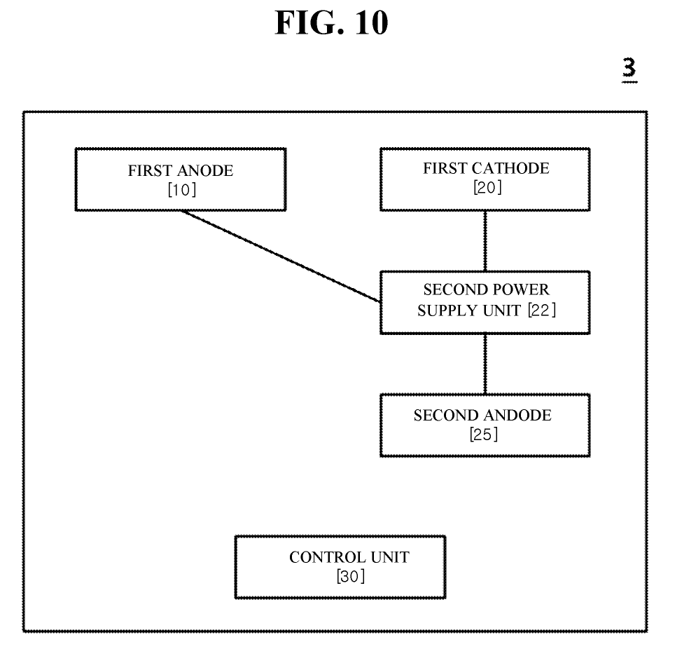

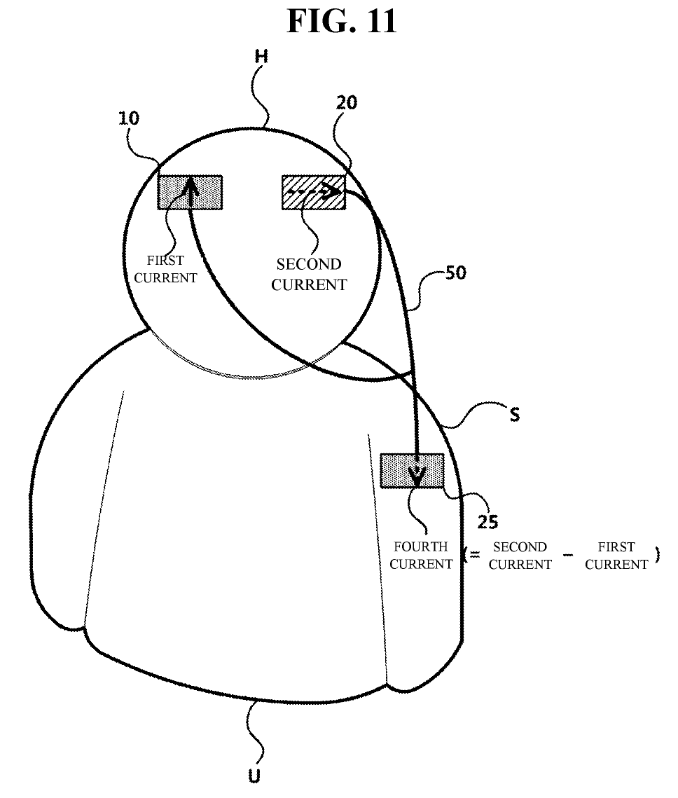

Hereinafter, a description will be given of a tCS apparatus 3 according to another embodiment of the inventive concept with reference to FIGS. 10 and 11. Herein, a difference between a tCS apparatus 2 according to another embodiment of the inventive concept and the tCS apparatus 3 according to another embodiment of the inventive concept is mainly described. It is apparent to those skilled in the art that technical features applied to the tCS apparatus 2 according to another embodiment of the inventive concept may be applied to the tCS apparatus 3 according to another embodiment of the inventive concept except for a described difference. Referring to FIG. 10, a drawing schematically illustrating a configuration of the tCS apparatus 3 according to another embodiment is shown. Referring to FIG. 11, a drawing illustrating the tCS apparatus 3 of FIG. 10, worn or attached to a user U is shown.

Meanwhile, in another embodiment, the tCS apparatus 3 may be a tACS apparatus which uses AC or a tDCS apparatus which uses DC.

Referring to FIG. 10, the tCS apparatus 3 may include a first anode 10, a first cathode 20, a second anode 25, a second power supply unit 22, and a control unit 30. In other words, in the tCS apparatus 3 according to another embodiment of the inventive concept, the number of anodes may be different from the number of cathodes. For example, the tCS apparatus 3 may include the two anodes 10 and 25 and the one cathode 20. All of the first anode 10, the first cathode 20, and the second anode 25 may be connected to the second power supply unit 22.

Referring to FIG. 11, if the user U wears or attaches the tCS apparatus 3 on or to a head H of the user U, the first anode 10 may come into contact with a first part of the head H. A first current may flow through the first anode 10. If the user U wears or attaches the tCS apparatus 3 on or to the head H, the first cathode 20 may come into contact with a second part of the head H. A second current which has a different amount of current from the first current may flow through the first cathode 20.

Further, the second anode 25 may be electrically connected with the first anode 10 and the first cathode 20 and, for example, may be located to come into contact with a body of the user U rather than the head H. A fourth current may flow through the second anode 25. The fourth current may have the same amount of current as an amount of current in which an amount of the first current is excluded from an amount of the second current.

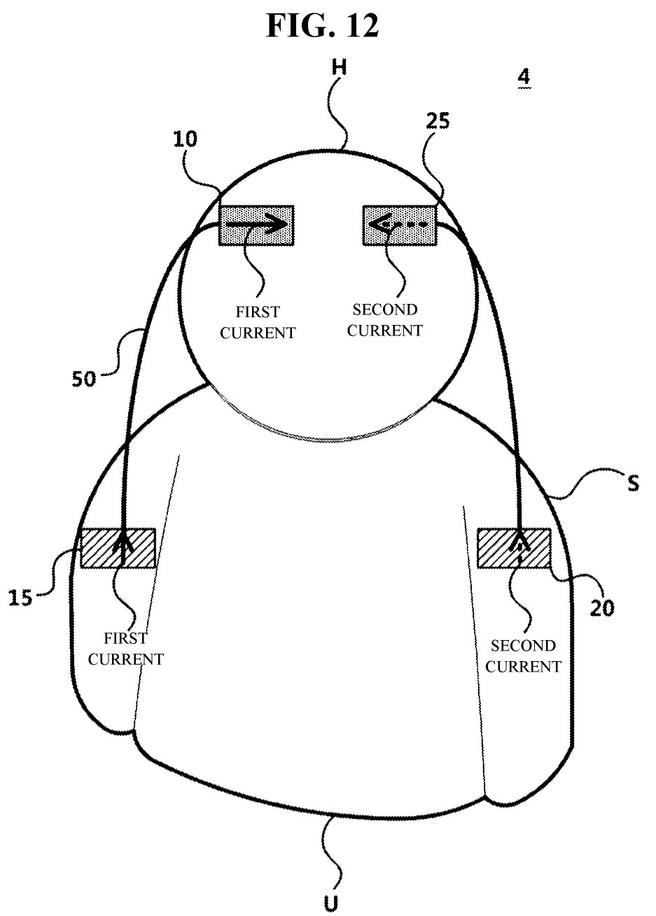

Hereinafter, a description will be given of a tCS apparatus 4 according to another embodiment of the inventive concept with reference to FIG. 12. Herein, a difference between a tCS apparatus 1 according to an embodiment of the inventive concept and the tCS apparatus 4 according to another embodiment of the inventive concept is mainly described. It is apparent to those skilled in the art that technical features applied to the tCS apparatus 1 according to an embodiment of the inventive concept may be applied to the tCS apparatus 4 according to another embodiment of the inventive concept except for a described difference. Referring to FIG. 12, a drawing schematically illustrating a configuration of the tCS apparatus 4 according to another embodiment is shown.

Meanwhile, in another embodiment, the tCS apparatus 4 may be a tACS apparatus which uses AC or a tDCS apparatus which uses DC.

Referring to FIG. 12, the tCS apparatus 4 according to another embodiment of the inventive concept may have the same configuration as the tCS apparatus 1 shown in FIG. 1. Herein, in the tCS apparatus 4 according to another embodiment of the inventive concept, the same type of electrodes may come into contact with a head H of a user U. For example, a first anode 10 and a second anode 25 may come into contact with a first part and a second part of the head H, respectively. In detail, the tCS apparatus 4 may include the first anode 10 which comes into contact with the first part of the head H and through which a first current flows and the second anode 25 which comes into contact with the second part of the head H and through which a second current having a different amount of current from the first current flows.

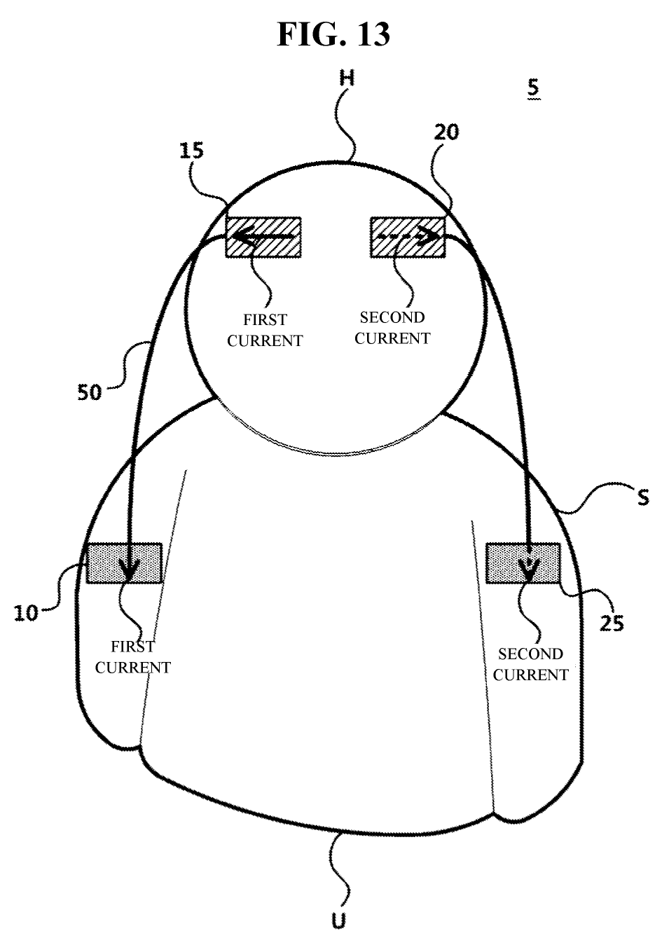

Hereinafter, a description will be given of a tCS apparatus 5 according to another embodiment of the inventive concept with reference to FIG. 13. Herein, a difference between a tCS apparatus 1 according to an embodiment of the inventive concept and the tCS apparatus 5 according to another embodiment of the inventive concept is mainly described. It is apparent to those skilled in the art that technical features applied to the tCS apparatus 1 according to an embodiment of the inventive concept may be applied to the tCS apparatus 5 according to another embodiment of the inventive concept except for a described difference. Referring to FIG. 13, a drawing schematically illustrating a configuration of the tCS apparatus 5 according to another embodiment is shown.

Meanwhile, in another embodiment, the tCS apparatus 5 may be a tACS apparatus which uses AC or a tDCS apparatus which uses DC.

Referring to FIG. 13, the tCS apparatus 5 according to another embodiment of the inventive concept may have the same configuration as the tCS apparatus 1 shown in FIG. 1. Herein, in the tCS apparatus 5 according to another embodiment of the inventive concept, the same type of electrodes may come into contact with a head H of a user U. For example, a second cathode 15 and a first cathode 20 may come into contact with a first part and a second part of the head H, respectively. In detail, the tCS apparatus 5 may include the second cathode 15 which comes into contact with the first part of the head H and through which a first current flows and the first cathode 20 which comes into contact with the second part of the head H and through which a second current having a different amount of current from the first current flows.

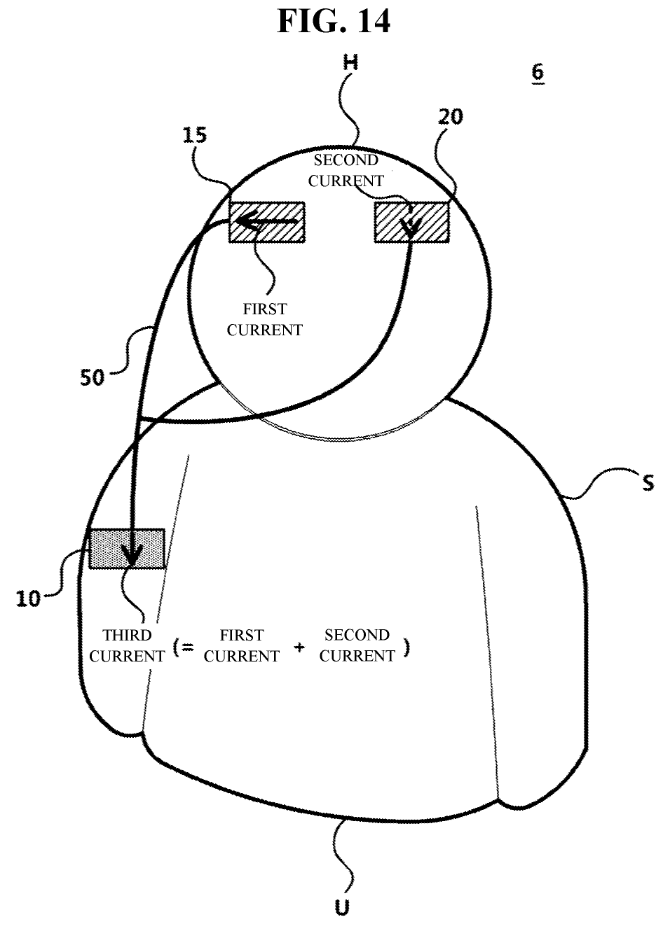

Hereinafter, a description will be given of a tCS apparatus 6 according to another embodiment of the inventive concept with reference to FIG. 14. Herein, a difference between a tCS apparatus 2 according to another embodiment of the inventive concept and the tCS apparatus 6 according to another embodiment of the inventive concept is mainly described. It is apparent to those skilled in the art that technical features applied to the tCS apparatus 2 according to another embodiment of the inventive concept may be applied to the tCS apparatus 6 according to another embodiment of the inventive concept except for a described difference. Referring to FIG. 14, a drawing schematically illustrating a configuration of the tCS apparatus 6 according to another embodiment is shown.

Meanwhile, in another embodiment, the tCS apparatus 6 may be a tACS apparatus which uses AC or a tDCS apparatus which uses DC.

Referring to FIG. 14, the tCS apparatus 6 according to another embodiment of the inventive concept may have the same configuration as the tCS apparatus 2 shown in FIG. 8. Herein, in the tCS apparatus 6 according to another embodiment of the inventive concept, the same type of electrodes may come into contact with a head H of a user U. For example, a second cathode 15 and a first cathode 20 may come into contact with a first part and a second part of the head H, respectively. In detail, the tCS apparatus 6 may include the second cathode 15 which comes into contact with the first part of the head H and through which a first current flows and the first cathode 20 which comes into contact with the second part of the head H and through which a second current having a different amount of current from the first current flows.

Meanwhile, a first anode 10 may be electrically connected with the second cathode 15 and the first cathode 15 and, for example, may be located to come into contact with a body of a user U except for the head H. A third current may flow through the first anode 10. The third current may have the same amount of current as an amount of current in which an amount of the first current is added to an amount of the second current.

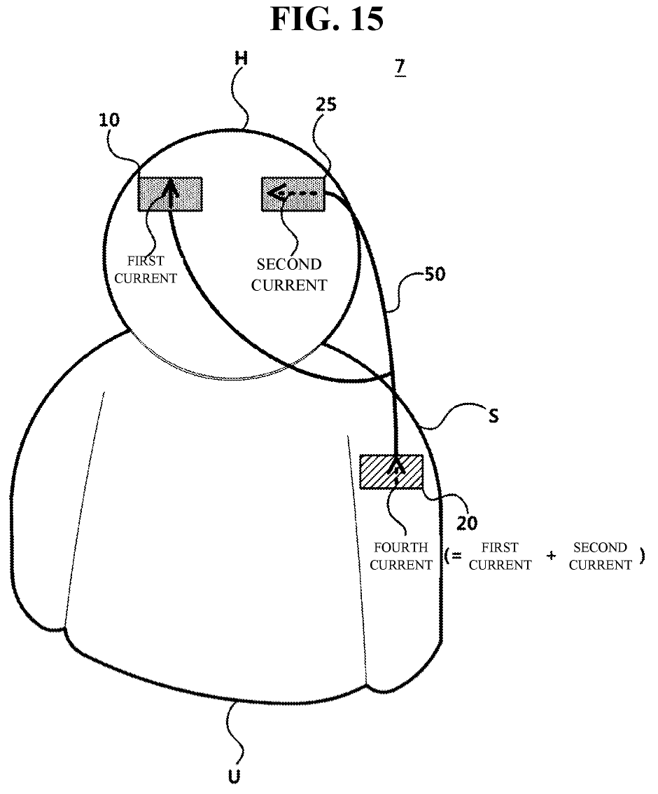

Hereinafter, a description will be given of a tCS apparatus 7 according to another embodiment of the inventive concept with reference to FIG. 15. Herein, a difference between a tCS apparatus 3 according to another embodiment of the inventive concept and the tCS apparatus 7 according to another embodiment of the inventive concept is mainly described. It is apparent to those skilled in the art that technical features applied to the tCS apparatus 3 according to another embodiment of the inventive concept may be applied to the tCS apparatus 7 according to another embodiment of the inventive concept except for a described difference. Referring to FIG. 15, a drawing schematically illustrating a configuration of the tCS apparatus 7 according to another embodiment is shown.

Meanwhile, in another embodiment, the tCS apparatus 7 may be a tACS apparatus which uses AC or a tDCS apparatus which uses DC.

Referring to FIG. 15, the tCS apparatus 7 according to another embodiment of the inventive concept may have the same configuration as the tCS apparatus 3 shown in FIG. 10. Herein, in the tCS apparatus 7 according to another embodiment of the inventive concept, the same type of electrodes may come into contact with a head H of a user U. For example, a first anode 10 and a second anode 25 may come into contact with a first part and a second part of the head H, respectively. In detail, the tCS apparatus 7 may include the first anode 10 which comes into contact with the first part of the head H and through which a first current flows and the second anode 25 which comes into contact with the second part of the head H and through which a second current having a different amount of current from the first current flows.

Meanwhile, a first cathode 20 may be electrically connected with the first anode 10 and the second anode 25 and, for example, may be located to come into contact with a body of a user U except for the head H. A fourth current may flow through the first cathode 20. The fourth current may have the same amount of current as an amount of current in which an amount of the first current is added to an amount of the second current.

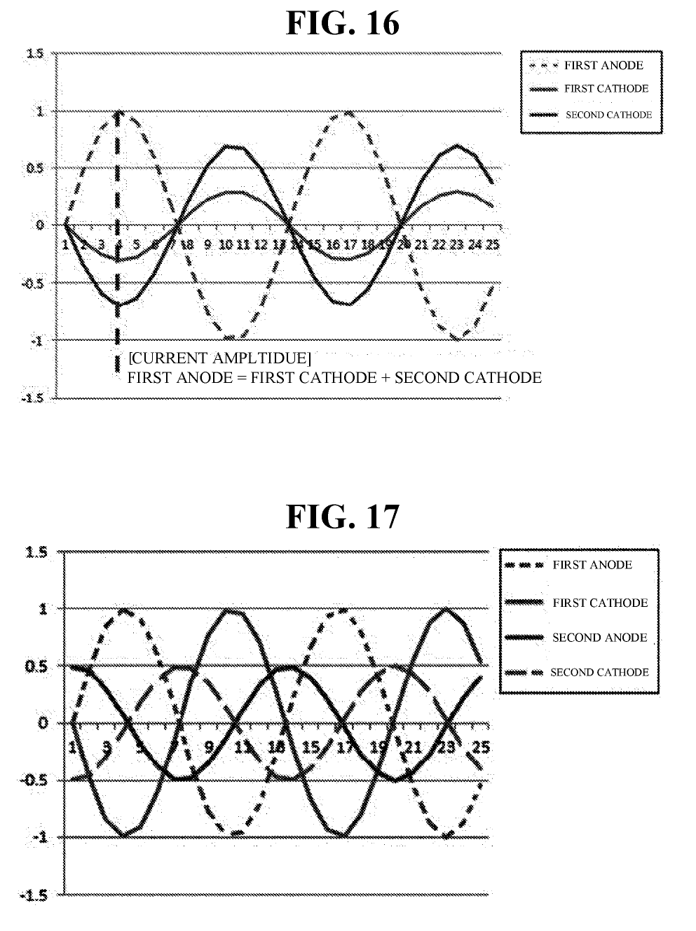

Hereinafter, a description will be given of a tCS apparatus according to another embodiment of the inventive concept with reference to FIGS. 16 and 17. Herein, a difference between a tCS apparatus 1 according to an embodiment of the inventive concept and the tCS apparatus according to another embodiment of the inventive concept is mainly described. It is apparent to those skilled in the art that technical features applied to the tCS apparatus 1 according to an embodiment of the inventive concept may be applied to the tCS apparatus according to another embodiment of the inventive concept except for a described difference. Referring to FIGS. 16 and 17, a drawing illustrating a current of the tCS apparatus according to another embodiment is shown.

Meanwhile, in another embodiment, the tCS apparatus may be a tACS apparatus which uses AC.

In the tCS apparatus which is worn or attached to a head H of a user U to provide electrical stimulation to a bran of the user U, the tCS apparatus may include a first electrode which comes into contact with a first part of the head H and through which a first current flows and a second electrode which comes into contact with a second part of the head H and through which a second current different from the first current flows. The first current and the second current may differ from each other in any one of amplitude, phase, and frequency.

The first electrode and the second electrode may come into contact with the first part and the second part, respectively. As shown in FIGS. 1 and 15, each of the first electrode and the second electrode may be an anode or a cathode. In other words, the first electrode and the second electrode may be any one of “anode and anode”, “anode and cathode”, “cathode and anode”, and “cathode and cathode”. Herein, as described above, the first current which flows through the first electrode and the second current which flows through the second electrode may differ from each other in at least one of amplitude, phase, and frequency.

For example, the first current and the second current may differ from each other in amplitude. Referring to FIGS. 9 and 16, the first electrode may be a first anode 10 and the second electrode may be a first cathode 20. Amplitude of the first current which flows through the first anode 10 may be the same as that of the second current which flows through the first cathode 20 and that of a third current which flows through a second cathode 15. Thus, the amplitude of the first current which flows through the first anode 10 may differ from the amplitude of the second current which flows through the first cathode 20.

Further, the first current and the second current may differ from each other in amplitude and phase. Referring to FIGS. 12 and 17, the first electrode may be a first anode 10 and the second electrode may be a second anode 25. The first current which flows through the first anode 10 may differ from the second current which flows through the second anode 25 in amplitude and phase.

According to embodiments of the inventive concept, a transcranial current stimulation (tCS) apparatus may stimulate a brain of a user through various networks of an inner brain by having different amounts of current which flows through an anode and a cathode.

According to embodiments of the inventive concept, the tCS apparatus may be safely used in a user's daily life by a general user who does not have professional knowledge.

According to embodiments of the inventive concept, the tCS apparatus may have portability and wearability due to its simple configuration.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A transcranial current stimulation (tCS) apparatus configured to be worn or attached to a head of a user to provide electrical stimulation a brain of the user, the tCS apparatus comprising:
a first anode configured to come into contact with a first part of the head such that a first current flows through the first anode; and
a first cathode configured to come into contact with a second part of the head such that a second current having a different amount of current from the first current flows through the first cathode.

2. The tCS apparatus of claim 1, further comprising:
a second cathode configured to be electrically connected with the first anode such that the first current flows through the second cathode; and
a second anode configured to be electrically connected with the first cathode such that the second current flows through the second anode.

3. The tCS apparatus of claim 2, wherein the second cathode and the second anode come into contact with a body of the user except for the head.

4. The tCS apparatus of claim 1, wherein the first anode is connected to a first power supply unit, and
wherein the first cathode is connected to a second power supply unit different from the first power supply unit.

5. The tCS apparatus of claim 1, wherein each of the first current and the second current has an amount of current which is greater than 0 mA and is less than or equal to 2 mA.

6. The tCS apparatus of claim 1, further comprising:
a second cathode configured to be electrically connected with the first anode and the first cathode such that a third current flows through the second cathode,
wherein the third current has the same amount of current as an amount of current in which an amount of the second current is excluded from an amount of the first current.

7. The tCS apparatus of claim 1, further comprising:
a second anode configured to be electrically connected with the first anode and the first cathode such that a fourth current flows through the second anode,
wherein the fourth current has the same amount of current as an amount of current in which an amount of the first current is excluded from an amount of the second current.

8. A transcranial current stimulation (tCS) apparatus configured to be worn or attached to a head of a user to provide electrical stimulation a brain of the user, the tCS apparatus comprising:
a first anode configured to come into contact with a first part of the head such that a first current flows through the first anode; and
a second anode configured to come into contact with a second part of the head such that a second current having a different amount of current from the first current flows through the second anode.

9. A transcranial current stimulation (tCS) apparatus configured to be worn or attached to a head of a user to provide electrical stimulation a brain of the user, the tCS apparatus comprising:
a first cathode configured to come into contact with a first part of the head such that a first current flows through the first cathode; and
a second cathode configured to come into contact with a second part of the head such that a second current having a different amount of current from the first current flows through the second cathode.

10. The tCS apparatus of claim 1, wherein the first current and the second current differ from each other in phase or frequency.

11. The tCS apparatus of claim 8, wherein the first current and the second current differ from each other in phase or frequency.

12. The tCS apparatus of claim 9, wherein the first current and the second current differ from each other in phase or frequency.
